SEQUENCE LISTING (45 sequences)

SEQ ID NO: 1 - DNA, 1556 bp, Homo sapiens
atggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgc

SEQ ID NO: 2 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 3 - DNA, 20 bp, Homo sapiens
tggtaaggccctgggcaggt

SEQ ID NO: 4 - RNA, 20 nt, Homo sapiens
ugguaaggcccugggcaggu

SEQ ID NO: 5 - DNA, 20 bp, Homo sapiens
ttctccacaggagtcagatg

SEQ ID NO: 6 - RNA, 20 nt, Homo sapiens
uucuccacaggagucagaug

SEQ ID NO: 7 - DNA, 20 bp, Homo sapiens
ttctccacaggagtcaggtg

SEQ ID NO: 8 - RNA, 20 nt, Homo sapiens
uucuccacaggagucaggug

SEQ ID NO: 9 - DNA, 20 bp, Homo sapiens
agatgcaccatggtgtctgt

SEQ ID NO: 10 - RNA, 20 nt, Homo sapiens
agaugcaccauggugucugu

SEQ ID NO: 11 - DNA, 20 bp, Homo sapiens
aggtgcaccatggtgtctgt

SEQ ID NO: 12 - RNA, 20 nt, Homo sapiens
aggugcaccauggugucugu

SEQ ID NO: 13 - DNA, 20 bp, Homo sapiens
tcctaaggagaagtctgccg

SEQ ID NO: 14 - RNA, 20 nt, Homo sapiens
uccuaaggagaagucugccg

SEQ ID NO: 15 - DNA, 29 bp, Homo sapiens
gacttctccacaggagtcagatgcaccat

SEQ ID NO: 16 - DNA, 29 bp, Homo sapiens
gaaggtggtaaggccctgggcaggttggt

SEQ ID NO: 17 - DNA, 29 bp, Homo sapiens
ctgactcctaaggagaagtctgccgttac

SEQ ID NO: 18 - protein, 7 aa, Homo sapiens
GGEALGR

SEQ ID NO: 19 - DNA, 27 bp, Homo sapiens
ggtggtgaggccctgggcaggttggta

SEQ ID NO: 20 - DNA, 27 bp, Homo sapiens
ggtggtaaggccctgggcaggttggta

SEQ ID NO: 21 - DNA, 23 bp, Homo sapiens
tggtaaggccctgggcaggttgg

SEQ ID NO: 22 - protein, 16 aa, Homo sapiens
MVHLTPEEKSAVTALW

SEQ ID NO: 23 - DNA, 60 bp, Homo sapiens
caaacagacaccatggtgcatctgactcctgaggagaagtctgccgttactgccctgtgg

SEQ ID NO: 24 - DNA, 60 bp, Homo sapiens
ccacagggcagtaacggcagacttctcctcaggagtcagatgcaccatggtgtctgtttg

SEQ ID NO: 25 - DNA, 60 bp, Homo sapiens
caaacagacaccatggtgcatctgactcctgtggagaagtctgccgttactgccctgtgg

SEQ ID NO: 26 - DNA, 60 bp, Homo sapiens
ccacagggcagtaacggcagacttctccacaggagtcagatgcaccatggtgtctgtttg

SEQ ID NO: 27 - DNA, 23 bp, Homo sapiens
agatgcaccatggtgtctgtttg

SEQ ID NO: 28 - DNA, 48 bp, Homo sapiens
atggtgcatctgactcctgtggagaagtctgccgttactgccctgtgg

SEQ ID NO: 29 - DNA, 45 bp, Homo sapiens
ccacagggcagtaacggcagacttctccacaggagtcagatgcac

SEQ ID NO: 30 - DNA, 23 bp, Homo sapiens
ttctccacaggagtcagatgcac

SEQ ID NO: 31 - protein, 16 aa, Homo sapiens
MVHLTPEEKSAVTALW

SEQ ID NO: 32 - DNA, 48 bp, Homo sapiens
atggtgcatctgactcctgaggagaagtctgccgttactgccctgtgg

SEQ ID NO: 33 - DNA, 48 bp, Homo sapiens
ccacagggcagtaacggcagacttctcctcaggagtcagatgcaccat

SEQ ID NO: 34 - DNA, 48 bp, Homo sapiens
atggtgcatctgactcctaaggagaagtctgccgttactgccctgtgg

SEQ ID NO: 35 - DNA, 48 bp, Homo sapiens
ccacagggcagtaacggcagacttctccttaggagtcagatgcaccat

SEQ ID NO: 36 - DNA, 23 bp, Homo sapiens
tcctaaggagaagtctgccgtta

SEQ ID NO: 37 - protein, 7 aa, Homo sapiens
GGEALGR

SEQ ID NO: 38 - DNA, 23 bp, Homo sapiens
tggtgaggccctgggcaggttgg

SEQ ID NO: 39 - DNA, 13 bp, Homo sapiens
gtggtgaggccct

SEQ ID NO: 40 - DNA, 13 bp, Homo sapiens
gtggtggggccct

SEQ ID NO: 41 - protein, 7 aa, Homo sapiens
GGEALGR

SEQ ID NO: 42 - DNA, 23 bp, Homo sapiens
tggtgaggccctgggcaggttgg

SEQ ID NO: 43 - DNA, 11 bp, Homo sapiens
tggtaaggccc

SEQ ID NO: 44 - DNA, 11 bp, Homo sapiens
tggtgaggccc

SEQ ID NO: 45 - DNA, 11 bp, Homo sapiens
tggtggggccc

(19) United States
(12) Patent Application Publication
(10) Pub. No.: US 2022/0033857 A1
(43) Pub. Date: Feb. 3, 2022

(54) EDITING OF HAEMOGLOBIN GENES

(71) Applicant: Oxford University Innovation Limited, Oxford (Oxfordshire) (GB)

(72) Inventors: James DAVIES, Oxford (Oxfordshire) (GB); Douglas HIGGS, Oxford (Oxfordshire) (GB); Mohsin BADAT, Oxford (Oxfordshire) (GB)

(21) Appl. No.: 17/278,679

(22) PCT Filed: Sep. 25, 2019
(86) PCT No.: PCT/GB2019/052696

§ 371 Date: Mar. 23, 2021

(30) Foreign Application Priority Data
Sep. 26, 2018 (GB) 1815670.3

(51) Int. Cl. C12N 15/90; A61K 35/28; C07K 14/805

(52) U.S. Cl. CPC C12N 15/907; C12N 2310/20; C07K 14/805; A61K 35/28

(57) ABSTRACT

The present invention relates to a process for producing a modified nucleic acid, wherein the nucleic acid comprises a mutant haemoglobin B (HBB) gene encoding a mutant Hb-β polypeptide. The process comprises using a base editor, preferably with a gRNA, to edit the mutant HBB gene to change a first (mutant) codon in that gene into a second, non-wild-type codon, wherein the Hb-β polypeptide encoded by that edited HBB gene has a non-wild-type, yet phenotypically-viable, amino acid sequence. The invention also provides a population of isolated haematopoietic stem cells, the stem cells comprising edited HBB genes.

The present invention relates to a process for producing a modified nucleic acid, wherein the nucleic acid comprises a mutant haemoglobin B (HBB) gene encoding a mutant Hb-β polypeptide. The process comprises using a base editor, preferably with a gRNA, to edit the mutant HBB gene to change a first (mutant) codon in that gene into a second, non-wild-type codon, wherein the Hb-β polypeptide encoded by that edited HBB gene has a non-wild-type, yet phenotypically-viable, amino acid sequence. The invention also provides a population of isolated haematopoietic stem cells, the stem cells comprising edited HBB genes.

Haemoglobin or hemoglobin (Hb) is a protein found in the red blood cells of all vertebrates except Channichthyidae and also in some invertebrates. Haemoglobin carries oxygen in the blood from the lungs or gills to the body tissues to allow aerobic respiration to take place. Haemoglobin also carries other gases such as carbon dioxide.

Haemoglobin consists of multiple globin chain subunits which associate with non-protein prosthetic heme groups. Heme groups consist of an iron ion in a porphyrin heterocyclic ring. The iron ion binds oxygen. The majority of haemoglobin protein in adult humans is in the form of haemoglobin A (HbA). This tetramer consists of two α (Hb-α1 and Hb-α2) and two β(Hb-β) subunits. The Hb-α1 and Hb-α2 subunits are coded for by the HBA1 and HBA2 genes, respectively. The Hb-β subunits are coded for by the HBB gene. A minority of haemoglobin in adult humans is in the form of haemoglobin A2 (HbA2) which consists of two α and two δ subunits. The most common form of haemoglobin in human at birth, which is also present as a minority in adults, is haemoglobin F (HbF) which consists of two α and two γ subunits. The amino acid sequences of haemoglobin chains differ between species and also within species. Within species, variants in haemoglobin chains may or may not cause disease.

Haemoglobinopathies are genetic defects resulting in abnormal structures of the haemoglobin subunits. Examples of haemoglobinopathies include thalassaemia and sickle cell disease.

Thalassaemias are a group of genetic blood disorders characterised by abnormal haemoglobin production. The symptoms of thalassaemia include anaemia and excess iron in the body. It is estimated that in 2013 there were 280 million people worldwide with thalassaemia. There were approximately 16,800 deaths resulting from thalassaemia in 2015. Thalassaemia is inherited in an autosomal recessive manner meaning that both parents must carry thalassaemia and they each pass this on to their child. Thalassaemia is caused by defective α or β haemoglobin chains causing production of abnormal red blood cells. Alpha-thalassaemia involves defects in the α chains. Beta-thalassaemia involves defects in the β chains. Defective β chains can either lead to a reduced quantity of functional haemoglobin being produced (β+) or no functional haemoglobin being produced (β0). Inheriting two β0 alleles leads to the most severe form of β-thalassemia, while inheriting one mutated allele and one normal allele may not produce any symptoms. Inheriting either one or two β+ alleles leads to an intermediate severity of disease. Thalassaemia is currently treated by regular blood transfusions, iron chelation, folic acid or bone marrow transplant.

Sickle cell disease is a group of genetic blood disorders characterised by rigid, sickle-like red blood cells. These cells are less able to deform to pass through capillaries leading to vessel occlusion and ischaemia. Sickled cells are destroyed by the body, but are replaced at a slower rate leading to anaemia. The symptoms of sickle cell disease include attacks of pain, increased risk of infection, anaemia and stroke. These symptoms can be managed with painkillers, antibiotics and blood transfusions. Haematopoietic stem cell or bone marrow transplants can potentially cure sickle cell disease, but these involve significant risk. In 2015, it is estimated that 4.4 million people around the world had sickle cell disease and approximately 114,800 deaths resulted from it.

One of the causes of thalassemia is Haemoglobin E. Haemoglobin E (HbE) is due to a point mutation in codon 27 of the human HBB gene. The wild type codon is GAG which codes for a glutamate residue. The codon in HbE is GAA which codes for a lysine residue. HbE has a high prevalence in parts of India, China and South East Asia (up to 70% of the population being carriers) because it confers protection against malaria which is also prevalent in these areas. The HbE variant reduces production of β-globin chains. When the HbE variant is inherited in combination with a β-thalassaemia mutation on the other allele of the HBB gene, severe HbE β-thalassaemia can develop. This combination causes approximately 50% of all severe thalassaemia worldwide which equates to approximately 20,000 births annually. These sufferers require blood transfusions every 2-3 weeks of their life.

Haemoglobin S (HbS) is a variant form of the HBB gene in which codon 7 is GTG (valine) rather than the wild type GAG (glutamate). HbS causes sickle cell disease either in homozygosity or in combination with either HbC or a thalassaemia mutation on the other allele.

A further haemoglobinopathy is Haemoglobin C (HbC) disease. This is a genetic disease in which the glutamic acid residue at position 7 of the β subunit is replaced with a lysine residue. Most sufferers do not have any symptoms, but symptoms can include spleen enlargement and haemolytic anaemia. Haemoglobin C is inherited in an autosomal recessive manner. Treatment is not usually required, but folic acid supplementation can help produce normal red blood cells and reduce the symptoms of resulting anaemia. However, when Haemoglobin C is co-inherited with Haemoglobin S on the other allele Haemoglobin SC disease results, which has a similar phenotype to sickle cell disease.

Some gene therapy approaches have recently been described to treat various haemoglobinopathies but these have significant safety concerns because an additional copy of the HBB gene is integrated randomly into the genome at thousands of different sites, leading to a potential for insertional mutagenesis and malignancy. These approaches are also likely to be less effective than the approach proposed herein because they do not correct the mutation in the genome leaving production of the abnormal globin chain intact. In addition, they are unable to use the full regulatory elements that are required for high levels of haemoglobin expression in contrast to our approach.

Several methods have been described for genome editing for the treatment of HbE beta thalassaemia and sickle cell disease including the following:

1. Conventional Cas9-induced deletions to reactivate expression of foetal haemoglobin either through mutagenesis of the promoters the beta globin genes or the BCL11A gene or its enhancer (which causes switching between foetal and adult forms of haemoglobin).
2. Deletion of the major regulatory element at the alpha globin gene (HBA) to reduce the toxicity caused by excess alpha globin chains in HbE beta thalassaemia.
3. Use of a DNA template and a conventional Cas9 cut at the beta globin gene, to correct the HbS and HbE mutations using the homology directed repair pathway, but this occurs at low efficiency and involves making double strand breaks in the DNA.

As mentioned above, some of these haemoglobinopathies can also be treated by regular blood transfusions. However, patients undergoing these procedures run the risk of iron overload; developing multiple transfusion related antibodies; hyperhaemolysis and infection from contaminated blood products. Bone marrow transplantation for haemoglobinopathies carries a risk of around 3% mortality for the best cases with sibling matches and this mortality rate rises to unacceptably high levels (>10%) by the age of 18. It would be desirable, therefore, to find further methods to treat and preferably to cure such patients.

The advent of gene-editing techniques such as CRISPR-Cas9 has meant that correcting the genetic defects underlying the above-mentioned haemoglobinopathies has become possible. However, the deleterious consequences of potential off-target effects are still a concern when using CRISPR-Cas9-based methods.

Base editing is a form of genetic editing in which one base pair is permanently converted to another base pair at a target locus. Base editors are guided to their target by an associated guide RNA. Unlike other methods of genetic editing, base editing does not introduce any double-strand DNA breaks into the target DNA; it does not require non-homologous end joining or homology-directed repair methods; and also it does not require any donor DNA templates. For these reasons, base editing can introduce specific point mutations more efficiently while introducing less off-target insertions, deletions, translocations and other modifications than other methods of gene editing such as CRISPR-Cas9. Base editing has been demonstrated in bacteria, yeast, plants, mammals and human embryos. Base editing can achieve transitions in genomic DNA from (C to T, A to G; which can be used to convert G to A and T to C on the opposite strand). Interconversion of purine to pyrimidine is not possible at present (i.e. C to G or A to T).

Base editors exhibit processivity and so can convert multiple bases within the single-strand DNA bubble created by Cas9. For this reason, base editors cannot be relied on to convert a single nucleotide polymorphism (SNP) associated with a disease exclusively to the wild type sequence. The number of different sequences that can result from use of a base editor depends on the number of bases that the editor targets within the editing window.

The most common programmable base editors (BEs) are BE3s which comprise a catalytically impaired CRISPR-Cas9 mutant which is incapable of making double-strand breaks; a single-strand-specific cytosine deaminase that converts C to U within a window of around five nucleotides in the single-strand DNA bubble created by the Cas9; a uracil glycosylase inhibitor that prevents uracil excision and downstream processes that reduce base editing efficiency and product purity; and nickase activity to nick the non-edited DNA strand which directs cellular DNA repair processes to replace the G-containing DNA strand and complete the C-G to T-A conversion.

Adenine base editors (ABEs) that convert A-T to G-C have only recently been developed (Gaudelli et al., 2017). A seventh-generation evolved ABE (i.e. ABE7.10) was shown to have a conversion efficiency of around 50% in human cells with a product purity of at least 99.9%, and an indel rate of 0.1% or lower. The ability of ABE7.10 to produce disease-suppressing mutations was tested by using it to make mutations in the promoters of two γ-globin genes (HBG1 and HBG2), as a model of enabling foetal haemoglobin production in adults for the treatment of sickle cell disease and thalassaemia, and to correct a mutation in the HFE gene for use in the treatment of the iron-storage disorder hereditary hemochromatosis. Notably, the authors of this paper (Gaudelli et al., 2017) did not try to use ABEs to correct any of the HbE, HbC or HbS mutations. The editing window for ABEs is a 4-base pair window. Hence all adenines in this 4-base pair window will be converted to guanines.

Liang (2017) discloses the use of a base editor to repair the HBB-28 (A>G) mutation. In patients having this mutation, the wild-type A at position −28 (in the ATA box upstream of the first exon) is replaced with G. Base editors BE, BE2 and BE3 were used to reverse this mutation.

Whilst WO2019/079347 refers to the use of therapeutic guide RNAs to treat beta-thalassemia, inter alia, the effective date of such disclosures is 16 Oct. 2018, which is after the priority date (and effective date) of the current patent application.

The inventors postulated that ABEs might be usable to correct mutations in certain other haemoglobinopathies in order to mitigate the deleterious effects of thalassaemias and sickle cell disease.

However, with regard to the HbE mutation (GAG→AAG at codon 27), the position of the PAM site (which is required for the gRNA to bind to the HBB gene) means that the 4-base pair window would cover both adenine (A) nucleotides. This would mean that the HbE codon (AAG=lysine) would not be changed back to the wild-type (GAG=glutamate); it would be changed to GGG (=glycine). The same issue would apply to the HbC mutation (GAG→AAG at codon 7), i.e. the use of an ABE would convert the mutated HBB codon (AAG=lysine) to GGG (=glycine).

With regard to HbS, the mutation in this case (GTG) does not comprise adenine. Whilst the non-coding strand codon—CAC—could be mutated to CGC, this would produce the codon GCG (alanine) in the coding strand, which is not the same as the wild-type (glutamate).

Therefore, ABEs cannot be used for correction of the HbE, HbC and HbS mutations.

The inventors had the insight to realise that it might not be necessary to correct the genetic defects in HbE, HbC and HbS patients in order to make them phenotypically normal; and hence that ABEs could be used to treat patients having HbE, HbC or HbS mutations if the mutant amino acids could be changed to phenotypically-viable amino acids (instead of the wild-type amino acids).

An extensive literature search uncovered a small number of reports where patients had been identified having normal blood phenotypes, but abnormal blood genotypes. In particular, an Hb Aubenas variant of HBB had been reported (Lacan et al., 1996) as having GGG (glycine) at codon 27, but having a normal blood phenotype. In addition, Blackwell et al. (1970) reported the results of a 1969 survey of blood samples from school children in Makassar, Indonesia, where starch-gel electrophoresis was used to try to find Hb variants. One male was identified with a HBB Glu7Ala mutation, but who was phenotypically normal. Furthermore, an Hb Lavagna variant of HBB as GGG (glycine) at codon 7, but a normal blood phenotype (personal communication).

It is therefore an object of the invention to provide a process for producing a modified nucleic acid, wherein the nucleic acid comprises a mutant haemoglobin B (HBB) gene encoding a mutant Hb-β polypeptide. The process comprises using a base editor, preferably with a gRNA, to edit the mutant HBB gene to change a first (mutant) codon in that gene into a second, non-wild-type codon, wherein the Hb-β polypeptide encoded by that edited HBB gene has a non-wild-type, yet phenotypically-viable, amino acid sequence. It is another object of the invention to provide a population of isolated haematopoietic stem cells, the stem cells comprising edited HBB genes.

In one embodiment, the invention provides a process for producing a modified nucleic acid molecule, the process comprising the steps:
(a) contacting a nucleic acid molecule comprising a mutant HBB gene encoding a mutant Hb-β polypeptide with a base editor, wherein the mutant HBB gene comprises a first non-wild-type codon coding for a first non-wild-type amino acid; and
(b) incubating the mutant HBB gene and base editor under conditions such that the base editor is targeted to the nucleotide sequence of the first non-wild-type codon and wherein the base editor edits one or more nucleotides in the first non-wild-type codon to produce a second non-wild-type codon which codes for a second non-wild-type amino acid, thereby producing a modified nucleic acid molecule comprising an edited HBB gene which encodes an edited Hb-β polypeptide,
   wherein the edited Hb-β polypeptide has a non-wild-type, yet phenotypically-viable, amino acid sequence.

Preferably, Step (a) comprises contacting a nucleic acid molecule comprising a mutant HBB gene encoding a mutant Hb-β polypeptide with a base editor and a gRNA, wherein the mutant HBB gene comprises a first non-wild-type codon coding for a first non-wild-type amino acid and wherein the gRNA is capable of targeting the base editor to the nucleotide sequence of the first non-wild-type codon of the mutant HBB gene; and Step (b) comprises incubating the mutant HBB gene, base editor and gRNA under conditions such that the gRNA targets the base editor to the nucleotide sequence of the first non-wild-type codon and wherein the base editor edits one or more nucleotides in the first non-wild-type codon to produce a second non-wild-type codon which codes for a second non-wild-type amino acid, thereby producing a modified nucleic acid molecule comprising an edited HBB gene.

In another embodiment, the invention provides a process for producing a modified nucleic acid molecule, the process comprising the steps:
(a) contacting a nucleic acid molecule comprising a mutant HBB gene encoding a mutant Hb-β polypeptide with a base editor and a gRNA, wherein the mutant HBB gene comprises a first non-wild-type codon coding for a first non-wild-type amino acid and wherein the gRNA is capable of targeting the base editor to the nucleotide sequence of the first non-wild-type codon of the mutant HBB gene; and
(b) incubating the mutant HBB gene, base editor and gRNA under conditions such that the gRNA targets the base editor to the nucleotide sequence of the first non-wild-type codon and wherein the base editor edits one or more nucleotides in the first non-wild-type codon to produce a second non-wild-type codon which codes for a second non-wild-type amino acid, thereby producing a modified nucleic acid molecule comprising an edited HBB gene which encodes an edited Hb-β polypeptide,
   wherein the edited Hb-β polypeptide has a non-wild-type, yet phenotypically-viable, amino acid sequence.

The invention also provides a population of isolated haematopoietic stem cells, the stem cells comprising HBB genes, the HBB genes comprising:

(i) a nucleotide sequence having 90-99.9% nucleotide sequence identity to SEQ ID NO: 1 or a nucleotide sequence encoding an amino acid sequence having 95-99.5% amino acid sequence identity to SEQ ID NO: 2; and wherein
(ii) the nucleotide sequence at the codon which corresponds to codon 7 in SEQ ID NO: 1 codes for glycine or alanine; and/or the nucleotide sequence at the codon which corresponds to codon 27 in SEQ ID NO: 1 codes for glycine.

In one embodiment, the invention provides a process for producing a modified nucleic acid molecule. The nucleic acid molecule is preferably a double-stranded DNA molecule. The nucleic acid molecule may be in the form of a linear or circular nucleic acid, e.g. a linear DNA fragment, a vector or plasmid. In other embodiments, the nucleic acid molecule is a chromosome. Preferably, the chromosome is a mammalian chromosome, more preferably a human chromosome.

In some preferred embodiments, the nucleic acid molecule is present in a cell, preferably a mammalian cell, and more preferably a human cell. Preferred cells include stem cells, e.g. haematopoietic stem cells. The haematopoietic stem cells may be foetal cells, juvenile cells or adult cells. In some embodiments, the stem cells are not embryonic stem cells.

The process of the invention comprises the step of (a) contacting a nucleic acid molecule comprising a mutant HBB gene with a base editor and a gRNA. As used herein, the term “contacting” includes bringing the mutant HBB gene, base editor and a gRNA together, e.g. in a suitable composition. This may be done in any suitable vessel, e.g. test tube, Eppendorf tube, tissue culture flask, etc.

Generally, the processes of the invention (particularly the contacting step and incubating step) will be carried out ex vivo or in vitro.

The nucleic acid molecule comprises a mutant HBB gene encoding a mutant Hb-β polypeptide. The HBB gene is preferably a mammalian gene, for example, a mouse, rat, cow, sheep, pig, horse, monkey or human gene. Most preferably, the HBB gene is a human gene.

The genomic DNA and amino acid sequences of the wild-type human HBB gene are given in SEQ ID NOs: 1-2.

The mutant HBB gene is termed herein as a “mutant” gene because it encodes a non-wild-type, i.e. mutant, Hb-β polypeptide.

As used herein, the term “wild-type” HBB gene refers to the HBB gene which is present in the majority of the members of that species (e.g. humans) and which encodes a non-mutant form of a Hb-β subunit.

Preferably, the mutant HBB gene consists of or comprises a nucleotide sequence which encodes an amino acid sequence having 90-99.5%, more preferably 95-99.5% sequence identity to SEQ ID NO: 2. Even more preferably, the mutant HBB gene consists of or comprises a nucleotide sequence which encodes an amino acid sequence having 97.0-99.5%, 97.5-99.5%, 98.0-99.5% or 99.0-99.5% sequence identity to SEQ ID NO: 2.

The mutant HBB gene comprises a first non-wild-type codon which encodes a first non-wild-type amino acid. The first non-wild-type amino acid is also referred to herein as the or a “mutant” amino acid. In many cases, the presence of this mutant amino acid in the Hb-β subunit polypeptide is the prime cause (preferably, the cause) for the diseased Hb phenotype. As used herein, the term “non-wild-type codon” means a codon at a defined position in the HBB gene which encodes an amino acid which is different to the amino acid which is present in the corresponding position of the wild-type HBB amino acid sequence (e.g. SEQ ID NO: 2). For the avoidance of any doubt, the term “first non-wild-type codon” does not refer merely to the wild-type codon which is first present in the HBB gene (e.g. when reviewing the nucleotide sequence in a 5′-3′ direction), although this might be the case in some embodiments.

Preferably, the mutant HBB gene consists of or comprises:

(i) a nucleotide sequence having 90-99.9% nucleotide sequence identity to SEQ ID NO: 1 or a nucleotide sequence encoding an amino acid sequence having 95-99.5% amino acid sequence identity to SEQ ID NO: 2; and wherein
(ii) the nucleotide sequence at the codon which corresponds to codon 7 in SEQ ID NO: 1 codes for lysine or valine; and/or the nucleotide sequence at the codon which corresponds to codon 27 in SEQ ID NO: 1 codes for lysine.

In some preferred embodiment, the mutant HBB gene consists of or comprises:

(i) a nucleotide sequence of SEQ ID NO: 1 or a nucleotide sequence encoding the amino acid sequence of SEQ ID NO: 2; apart from
(ii) the nucleotide sequence at the codon which corresponds to codon 7 in SEQ ID NO: 1 which codes for lysine or valine; and/or the nucleotide sequence at the codon which corresponds to codon 27 in SEQ ID NO: 1 which codes for lysine.

Preferably, the nucleotide sequence at the first non-wild-type codon which corresponds to codon 7 in SEQ ID NO: 1 is AAG or GTG; and/or the nucleotide sequence at the first non-wild-type codon which corresponds to codon 27 in SEQ ID NO: 1 is AAG.

The nucleic acid molecule comprising a mutant HBB gene is contacted with a base editor. As used herein, the term “base editor” refers to an enzyme which is capable of binding to a specific DNA sequence and can chemically convert nucleotides of one specific type in a DNA molecule to a different specific type (e.g. C to T or A to G, resulting in G to A or T to C on the opposite strand). These usually comprise Cas9 linked to a base editor protein such as the APOBEC or Adenine Base Editor protein, but other programmable nucleic acid binding proteins could be used.

In some embodiments, the base editor is a programmable nucleic acid binding protein (e.g. an impaired CRISPR-Cas9 mutant) which is capable of being targeted to a target (DNA) sequence. In some embodiments, the base editor is an enzyme which comprises a catalytically impaired CRISPR-Cas9 mutant which is incapable of making double-strand breaks.

Base editors are enzymes that combine programmable nucleic acid binding with an ability to change the nucleic acid bases at the target sequence. To date, base editors have been described that deaminate cytosine resulting in conversion to thymine or deaminate adenine resulting in conversion to guanine.

Examples of base editors include cytosine deaminating editors (C:G to T:A), e.g. AID-CRISPR-Cas9 (Nishida et al., 2016), BE3 (Komor et al., 2016), BE4 and BE-Gam (Komor et al., 2017), BE4max (Koblan et al., 2018) and AncBE4max (Koblan et al., 2018). Other examples of base editors include adenine deaminating editors (A:T to G:C). Preferably, the base editor is an adenine base editor (ABE). These convert A:T to G:C. Examples of preferred ABEs include Cas9-ABE7.10 ((Gaudelli et al., 2017); US 2018/0073012), xCas9-ABE7.10 (Hu et al., 2018) and ABEmax (Koblan et al., 2018).

The nucleic acid molecule comprising a mutant HBB gene is preferably also contacted with a gRNA. The function of the gRNA is to target the base editor to the nucleotide sequence of the first non-wild-type codon of the mutant HBB gene. The gRNA is therefore one which is capable of binding to a cognate base editor. In one embodiment, a gRNA is a chimeric RNA which is formed from a crRNA and a tracrRNA such as those which have been used in CRISPR/Cas systems (Jinek et al., 2012). The term gRNA is well accepted in the art. In some embodiments, wherein the base editor comprises Cas9 or an analogue or a variant thereof, the gRNA is a RNA which is capable of binding to Cas9, or to analogues or variants thereof.

The gRNA is generally made up of the ribonucleotides A, G, C and U. Modified ribonucleotides, deoxyribonucleotides, other synthetic bases and synthetic backbone linkages (such as peptide nucleic acid (PNA), locked nucleic acid (LNA), etc.) may also be used.

The gRNA comprises a targeting RNA sequence. The targeting sequence has a degree of sequence identity with the region of DNA in the HBB gene which includes the first non-wild-type codon (i.e. the target nucleic acid sequence). Preferably, the degree of sequence identity between the targeting RNA sequence and the target nucleic acid sequence is at least 80%, more preferably at least 90%, 95%, 99% or 100%. Preferably, the targeting RNA sequence is 14-30 nucleotides, more preferably 20-30 nucleotides in length.

In some embodiments of the invention, the nucleotide sequence of the PAM site in the target DNA is one which has been modified compared to the wild-type PAM nucleotide sequence in order to increase efficiency of the base-editing process. Such a modification is one which does not affect the function of the HBB polypeptide.

Preferably, the guide RNA sequence for editing codon 7 in an HbS patient is an 18-22 nucleotide guide RNA which is complementary to a nucleotide sequence located in SEQ ID NO: 15, wherein the wild-type complement of codon 7 (CTC) is replaced by CAC.

Preferably, the guide RNA sequence for editing codon 27 in an HbE patient is an 18-22 nucleotide guide RNA which is located in SEQ ID NO: 16, wherein the wild-type codon 27 (GAG) is replaced by AAG.

Preferably, the guide RNA target sequence for editing codon 7 in an HbC patient is an 18-22 nucleotide guide RNA which is located in SEQ ID NO: 17, wherein the wild-type of codon 7 (GAG) is replaced by AAG.

The preferred guide RNA target sequence for ABE7.10 for editing codon 27 to GGG is TGGTAAGGCCCTGGGCAGGT (SEQ ID NO: 3; the PAM sequence is TGG.), i.e. the RNA sequence is UGGUAAGGCCCUGGGCAGGU (SEQ ID NO: 4; the PAM sequence is TGG).

The preferred guide RNA target sequence for xCas9 ABE7.10 for editing codon 7 to GCG is TTCTCCACAGGAGTCAGATG (SEQ ID NO: 5; the PAM sequence is CAC), i.e. the RNA sequence is UUCUCCACAGGAGUCAGAUG (SEQ ID NO: 6, the PAM sequence is CAC).

The preferred guide RNA for the SNP rs713040 is TTCTCCACAGGAGTCAGGTG (SEQ ID NO: 7; the PAM sequence is CAC), i.e. the RNA sequence is UUCUCCACAGGAGTCAGGUG (SEQ ID NO: 8).

The preferred guide RNA target sequence for xCas9 ABE7.10 for generating an improved binding sequence for editing the PAM sequence to a more favourable sequence for the base editor for editing the HbS mutation is AGATGCACCATGGTGTCTGT (SEQ ID NO: 9; the PAM sequence is TTG), i.e. the RNA sequence is AGAUGCACCAUGGUGUCUGU (SEQ ID NO: 10). The variant of this sequence to account for rs713040 is AGGTGCACCATGGTGTCTGT (SEQ ID NO: 11; the PAM sequence is TTG), i.e. the RNA sequence is AGGUGCACCAUGGUGUCUGU (SEQ ID NO: 12).

The preferred guide RNA target sequence for xCas9 ABE7.10 for editing the HbC gene is TCCTAAGGAGAAGTCTGCCG (SEQ ID NO: 13; the PAM sequence is TTA), i.e. the RNA sequence is UCCUAAGGAGAAGUCUGCCG (SEQ ID NO: 14).

The above gRNA sequences can be shifted in either direction up to 4 bases. In addition, the length of the gRNA could be varied in length by +/−3 base pairs. In some embodiments, a second gRNA is used to increase the base-editing efficiency.

The mutant HBB gene, base editor and gRNA (when present) are incubated under conditions such that the base editor is targeted (preferably by the gRNA) to the nucleotide sequence of the first non-wild-type codon and wherein the base editor edits one or more (e.g. 1, 2 or 3) nucleotides in the first non-wild-type codon to produce a second non-wild-type codon which codes for a second non-wild-type amino acid, thereby producing a modified nucleic acid molecule comprising an edited HBB gene.

Suitable conditions for the base-editing are readily known in the art (e.g. (Gaudelli et al., 2017). In particular, procedures which are used for CRISPR/Cas9 (e.g. Genome Editing and Engineering: From TALENs, ZFNs and CRISPRs to Molecular Surgery, 2018, Ed. Krishnarao Appasani, Cambridge University Press; and references therein) may be adapted for use in the processes disclosed herein.

There are several ways in which base editors can be delivered, including:
a) DNA (e.g. in plasmid form)
b) mRNA of the base editor and synthetic guide RNA
c) Protein-gRNA complex or
d) Viral transduction

The machinery may be introduced into the cells using the following methods, inter alia:
a) Electroporation
b) Lipofection
c) Viral transduction or
d) Nanoparticles.

Once the base editor has been targeted to the first non-wild-type codon (preferably by the gRNA), the base editor edits one or more nucleotides in the first non-wild-type codon to produce a second non-wild-type codon which codes for a second non-wild-type amino acid.

During the editing process, the base editor (e.g. one comprising Cas9 or a variant or analogue thereof) will separate the two strands of the double-stranded target DNA (i.e. HBB gene). Base editors exhibit processivity and so such editors may convert more than one nucleotide within the single-strand DNA bubble. These one or more nucleotides may be present in the same codon or in adjacent codons (i.e. more than one codon may be edited).

The second non-wild-type codon encodes a second amino acid. The first and second amino acids are not the same. The second non-wild-type codon will be at the same position in the HBB gene (e.g. codon 7 or codon 27) as the first non-wild-type codon. Silent codon changes (which do not produce a change in amino acid) are excluded from the invention.

The base editor may edit one, two or three of the nucleotides in the first non-wild-type codon. Preferably, the base editor edits one or two nucleotides.

Some preferred examples of second non-wild-type codons, coding for non-wild-type amino acids, are given below based on the human HBB gene.

TABLE 1
Examples of haemoglobinopathies, mutant codons and edited codons

| Genotype | HbC | HbS | HbE |
|---|---|---|---|
| HBB codon number | 7 | 7 | 27 |
| Wild-type amino acid | glutamate (GAG) | glutamate (GAG) | Glutamate (GAG) |
| (codon) | | | |
| First non-wt amino | lysine (AAG) | valine (GTG) | lysine (AAG) |
| acid/mutant (codon) | | | |
| Second non-wt amino | glycine (GGG) | alanine (GCG) | glycine (GGG) |
| acid (codon) | | | |

As can be seen from the above table, the second non-wild-type codon is not the same as the wild-type codon, i.e. the editing process of the invention does not primarily result in a correction of the codon to the wild-type codon.

In most embodiments of the invention, the process will not be carried out on a single nucleic acid molecule; in general, the process will be applied to a population of nucleic acid molecules, which may be present within a population of cells. Within this population of nucleic acid molecules/cells, depending on the codon in question and the targeted nucleotide sequence, the base editor may edit different numbers of nucleotides within a single codon. In particular, due to the processivity of base editors, occurrences of 2 or 3 relevant nucleotides within the codon are more likely all to be edited.

For example, with regard to the HbE mutation (AAG=lysine), the use of an adenine base editor on this codon may produce a combination of up to 4 different results, depending on the reaction conditions:

| | |
|---|---|
| AAG (lysine = mutant) + ABE22→ | GAG (correction to wild-type codon) |
| | AGG (arginine = mutant) |
| | GGG (glycine = viable amino acid) |
| | AAG (lysine, no reaction, no editing) |

Preferably, the process of the invention is carried out under conditions which favour the editing of a first non-wild-type codon coding for a first non-wild-type amino acid to a second non-wild-type codon coding for a second non-wild-type amino acid.

In particular, there is provided a process of the invention wherein the process is applied to a population of nucleic acid molecules, vectors or cells and wherein at least 30%, 40%, 50%, 60%, 70%, 80% or 90% of the first non-wild-type codons coding for first non-wild-type amino acids in the nucleic acid molecules in the population of nucleic acid molecules, vectors or cells have been edited to second non-wild-type codons coding for second non-wild-type amino acids.

In this way, a modified nucleic acid comprising an edited HBB gene is produced. The edited

HBB gene is one which comprises the second non-wild-type codon coding for a second non-wild-type amino acid.

Preferably, the edited HBB gene consists of or comprises:

(i) a nucleotide sequence having 90-99.9% nucleotide sequence identity to SEQ ID NO: 1 or a nucleotide sequence encoding an amino acid sequence having 95-99.5% amino acid sequence identity to SEQ ID NO: 2; and wherein
(ii) the nucleotide sequence at the codon which corresponds to codon 7 in SEQ ID NO: 1 codes for glycine or alanine; and/or the nucleotide sequence at the codon which corresponds to codon 27 in SEQ ID NO: 1 codes for glycine.

In some preferred embodiments, the edited HBB gene consists of or comprises:

(i) a nucleotide sequence of SEQ ID NO: 1 or a nucleotide sequence encoding an amino acid sequence of SEQ ID NO: 2; apart from
(ii) the nucleotide sequence at the codon which corresponds to codon 7 in SEQ ID NO: 1 which codes for glycine or alanine; and/or the nucleotide sequence at the codon which corresponds to codon 27 in SEQ ID NO: 1 which codes for glycine.

Preferably, in the edited HBB gene, the nucleotide sequence at the codon which corresponds to codon 7 in SEQ ID NO: 1 is GGG or GCG; and/or the nucleotide sequence at the codon which corresponds to codon 27 in SEQ ID NO: 1 is GGG.

The second non-wild-type codon codes for a second non-wild-type amino acid, but not all non-wild-type amino acids will be capable of reversing or mitigated the phenotypic effect of the first non-wild type (i.e. mutant) amino acid.

The Hb-β polypeptide encoded by the edited HBB gene has a non-wild-type, yet phenotypically-viable, amino acid sequence. Preferably, the second non-wild-type amino acid is a phenotypically-viable amino acid.

Phenotypic viability of Hb-β polypeptides and amino acids may be tested at one or more different levels:

a) The clinical phenotype (i.e. the clinical symptoms/disease, e.g. anaemia, haemolysis);
b) The cellular phenotype (e.g. expression of the polypeptide or HbA tetramer in cells); and
c) The phenotype of the polypeptide (e.g. properties of the isolated HBB polypeptide).

In patients heterozygous for HbE mutations in combination with a wild type beta chain on the other allele, the level of mutant Hb-β polypeptides which are produced by their red blood cells only account for 27-30% of the total Hb-β polypeptides.

The phenotypic viability of the edited Hb-β polypeptide may therefore be tested in a cultured cells (e.g. CD34⁺ stem cells, e.g. HUDEP-2 cells) which express both the edited Hb-β polypeptide and a wild-type Hb-β polypeptide, and assessing the proportions of the two polypeptides (e.g. by HPLC). Such expression may be achieved by mutating one allele of the genomic HBB gene in cultured cells in the same manner as the base editor and leaving the other allele encoding the wild-type HBB sequence. An edited Hb-β polypeptide which is produced in such a system at a level which is at least 35% of the level of the total Hb-β polypeptide produced would be considered to be phenotypically viable.

In one embodiment, therefore, the expression level of the edited Hb-β polypeptide is at least 35% of the level of the total Hb-β polypeptide produced when both edited Hb-β polypeptides and wild-type Hb-β polypeptides are expressed in the same cells.

Preferably, the cells are primary cells differentiated from a CD34⁺ stem cell line in culture, or alternatively differentiated from using the HUDEP-2 cell line. Preferably, the level of expression of Hb-β polypeptides is determined by HPLC. Preferably, the level of expression of the edited Hb-β polypeptide is least 35%, more preferably at least 40%, and most preferably at least 50%, of the level of the total Hb-β polypeptide. Suitable conditions for the expression of the Hb-β polypeptides and the measurements thereof may be found in (Kurita et al., 2013; Trakarnsanga et al., 2017; Old et al., 2012; Mettananda et al., 2017).

The red blood cells of patients having the HbS mutation or the HbSC mutations have a sickle (i.e. crescent) shape which can readily be detected under the microscope.

A further test for such sickle cells is the sickle cell solubility test (Diggs and Walker, (1973) “A Solubility Test for Sickle Cell Hemoglobin: I. Aggregation and Separation of Soluble and Insoluble Components without Centrifugation”, Laboratory Medicine, Volume 4, Issue 10, 1 Oct. 1973, p. 27-31). This involves mixing a sample of the patient's blood with a sodium dithionite solution. A cloudy solution is indicative of the presence of sickle cells in the blood sample.

The phenotypic viability of the edited Hb-β polypeptide may therefore be tested in cultured primary cells (e.g. red cells differentiated from primary CD34⁺ stem cells) or a cell line (e.g. HUDEP-2 cells) which expresses the edited Hb-β polypeptide (either mono-allelically or bi-allelically) by differentiating the cells into red blood cells, and examining the red blood cells under the microscope. Such expression may be achieved by mutating one or both alleles of the genomic HBB gene in the cell line in the same manner as the base editor. The production of red blood cells having a wild-type or control (i.e. round or non-sickle-shaped) appearance would be considered as indicating that the edited Hb-β polypeptide is phenotypically viable.

In one embodiment, therefore, the ex vivo cultured cells which express the edited Hb-β polypeptide (either mono-allelically or bi-allelically) and which have differentiated into red blood cells have a wild-type (round) appearance. Preferably, the cultured erythroid cells derived from CD34⁺ stem cells or alternatively HUDEP-2 cells. Suitable conditions for the expression of the Hb-β polypeptide in primary cells and HUDEP-2 cell lines, differentiation to red blood cells and the microscopic examination of sickle cells may be found in (Kurita et al., 2013; Trakarnsanga et al., 2017; Old et al., 2012; Mettananda et al., 2017).

In some preferred embodiments, the position of the first non-wild-type codon is codon 7, the wild-type codon at this position is GAG (glutamate), the first non-wild-type (mutant) codon sequence is AAG (lysine), the base editor is an adenine base editor and the second non-wild-type codon is GGG (glycine). The rest of the Hb-β polypeptide has a wild-type sequence. Patients having this variant have been reported to have a normal blood phenotype (personal communication).

In some other preferred embodiments, the position of the first non-wild-type codon is codon 7, the wild-type codon at this position is GAG (glutamate), the first non-wild-type (mutant) codon sequence is GTG (valine), the base editor is an adenine base editor and the second non-wild-type codon is GCG (alanine). The rest of the Hb-β polypeptide has a wild-type sequence. In this case, the ABE acts on the non-coding strand to change CAC to CGC which then effects the desired change to GCG in the coding strand. Patients having this variant have been reported to have a normal blood phenotype (Blackwell et al., 1970; Viprakasit et al., 2002).

In other preferred embodiments, the position of the first non-wild-type codon is codon 27, the wild-type codon at this position is GAG (glutamate), the first non-wild-type (mutant) codon sequence is AAG (lysine), the base editor is an adenine base editor and the second non-wild-type codon is GGG (glycine). The rest of the Hb-β polypeptide has a wild-type sequence. Patients having this variant have been reported to have a normal blood phenotype (Lacan et al., 1996).

In yet other embodiments, the process additionally comprises, prior to Step (a), the step of obtaining a sample of haematopoietic stem cells from a subject, preferably from a human subject, wherein the stem cells comprise nucleic acid molecules comprising mutant HBB genes.

In other embodiments of the invention, the process additionally comprises, prior to Step (a), the step of modifying the nucleotide sequences of one or more PAM sites in the vicinity of the first non-wild-type codon in order to increase efficiency of the base-editing process.

In yet other embodiments, the process is performed on haematopoietic stem cells which have previously been obtained from a first subject, preferably from a human subject, wherein the stem cells comprise nucleic acid molecules comprising mutant HBB genes, the process additionally comprises the subsequent step of introducing a population of haematopoietic stem cells comprising modified nucleic acid molecules comprising edited HBB genes, optionally after expansion of the cells, into a second subject. Preferably, the first and second subjects are the same subjects (autologous transplantation) or related subjects (e.g. wherein the first subject is a sibling, parent, grandparent or first cousin of the second subject or vice versa). The haematopoietic stem cells may, for example, be foetal cells, juvenile cells or adult cells.

In yet a further embodiment, there is provided a population of isolated cells comprising haematopoietic stem cells or progenitor cells comprising edited HBB genes (e.g. in their chromosomes), the edited HBB genes comprising:

(i) a nucleotide sequence having 90-99.9% nucleotide sequence identity to SEQ ID NO: 1 or a nucleotide sequence encoding an amino acid sequence having 95-99.5% amino acid sequence identity to SEQ ID NO: 2; and wherein
(ii) the nucleotide sequence at the codon which corresponds to codon 7 in SEQ ID NO: 1 codes for glycine or alanine; and/or the nucleotide sequence at the codon which corresponds to codon 27 in SEQ ID NO: 1 codes for glycine.

The population of isolated cells may comprise at least 20% haematopoietic stem cells or progenitor cells having modified nucleic acid molecules comprising edited HBB genes, preferably at least 40%, at least 60%, at least 80% or 100% haematopoietic stem cells or progenitor cells having modified nucleic acid molecules comprising edited HBB genes. The remaining cells in the population of cells may comprise haematopoietic stem cells or progenitor cells having nucleic acid molecules comprising mutant or wild-type HBB genes.

The population of cells is preferably obtained from one of the following sources:
a) Cord blood collected at birth from the umbilical cord and placenta;
b) Bone marrow harvested directly from a patient;
c) Peripheral blood stem cells (e.g. collected by apheresis following administration of plerixafor or GCSF or chemotherapy); or
d) An identical twin, twin transplant or sibling to the patient.

In a further embodiment, there is provided a mixed population of haematopoietic stem cells or progenitor cells, the mixed population comprising:

(i) a population of haematopoietic stem cells or progenitor cells of the invention, wherein the cells comprise edited HBB genes (e.g. in their chromosomes); and
(ii) a population of haematopoietic stem cells or progenitor cells, wherein the cells comprise mutant or wild-type HBB genes (e.g. in their chromosomes).

In some preferred embodiments, the edited HBB gene consists of or comprises:

(i) a nucleotide sequence of SEQ ID NO: 1 or a nucleotide sequence encoding an amino acid sequence of SEQ ID NO: 2; except for
(ii) the nucleotide sequence at the codon which corresponds to codon 7 in SEQ ID NO: 1 which codes for glycine or alanine; and/or the nucleotide sequence at the codon which corresponds to codon 27 in SEQ ID NO: 1 which codes for glycine.

Preferably, the nucleotide sequence at the codon which corresponds to codon 7 in SEQ ID NO: 1 is GGG or GCG; and/or the nucleotide sequence at the codon which corresponds to codon 27 in SEQ ID NO: 1 is GGG.

The haematopoietic stem cells may, for example, be foetal cells, juvenile cells or adult cells.

The disclosure of each reference set forth herein is specifically incorporated herein by reference in its entirety.

### BRIEF DESCRIPTION OF THE FIGURES

FIG. 1. Haemoglobin E is caused by a mutation of codon 27 of the beta globin gene (GAG to AAG). This mutation can be corrected to its canonical sequence using Cas9-ABE7.10 or ABEmax and the guide RNA shown. This enzyme has a 4 bp window and shows processivity meaning that the base will not be corrected simply back to its canonical sequence—it is likely that most cells will be corrected to GGG, which results in the variant haemoglobin Hb Aubenas, which has a normal phenotype. Another variant haemoglobin is also possible but less likely (Hb R27).

FIG. 2. Sickle cell disease results in conversion of GAG (Glutamate) at position 7 to GTG (Valine). This can be corrected by base editor xCas9-ABE7.10 to GCG (which encodes HbG-Makassar) through editing the Adenine on the opposite strand to Guanine. Alanine at position 7 is described in the literature as HbG-Makassar, which has a normal phenotype. It is likely editing efficiency could be improved by mutating the codon 2 from GTG (valine) to GCG (alanine) which makes a more efficient protospacer active motif (PAM), for the editing of the HbS mutation. Both of these guide RNAs could be used simultaneously.

FIG. 3. Haemoglobin C (which is the third most important disease causing variant) could also be corrected using base editors to another variant haemoglobin (Hb Lavagna) that has a normal phenotype.

FIGS. 4A and 4B. Experimental overview of production of the Hb Aubenas variant in wild type CD34⁺ human haemopoietic stem and progenitor cells using ABEmax.

FIG. 5. Creation of over 50% editing of wild type codon 27 from glutamate to glycine (Hb Aubenas).

FIG. 6. Editing of haemopoietic stem cells from patients with HbE-beta0 thalassaemia.

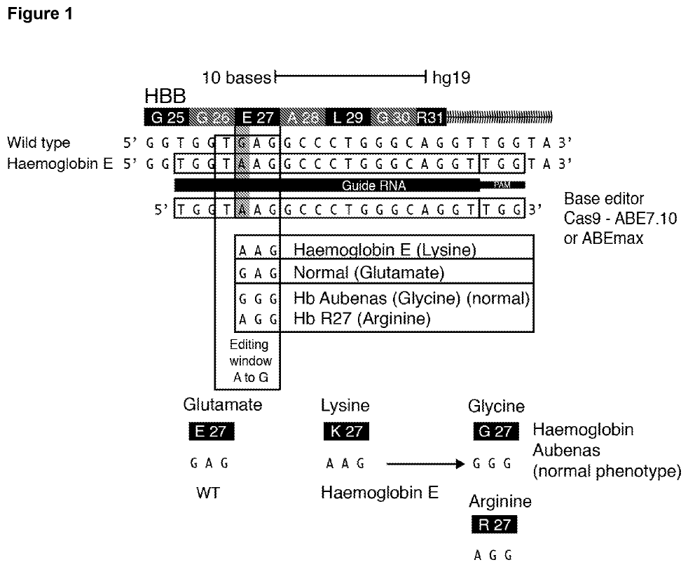

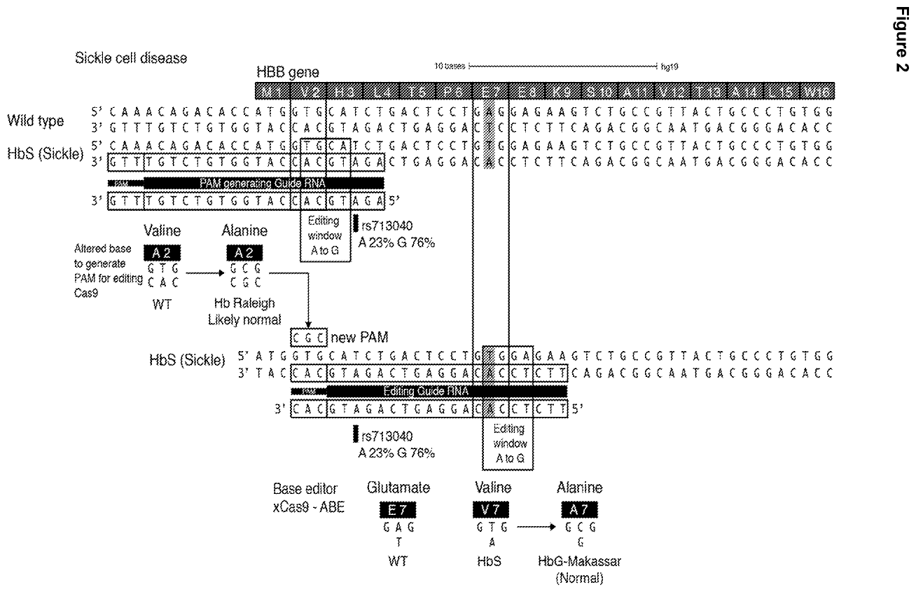

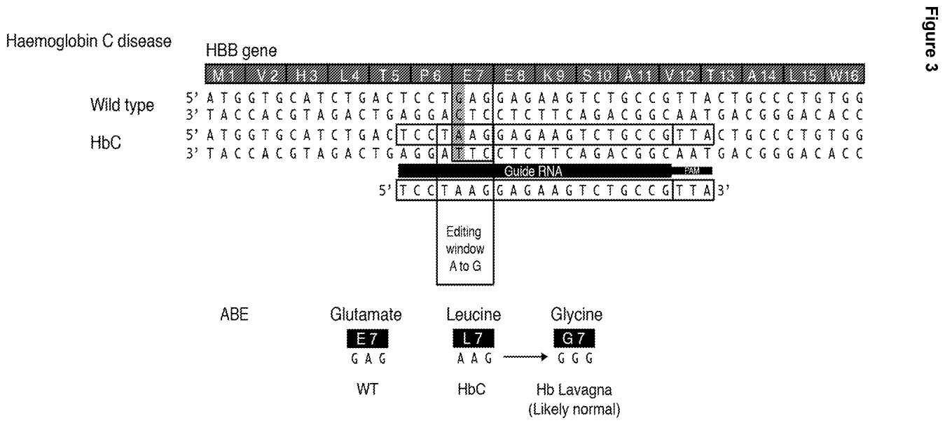

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Production of the Hb Aubenas Variant in Wild Type CD34+ Human Haemopoietic Stem and Progenitor Cells Using ABEmax

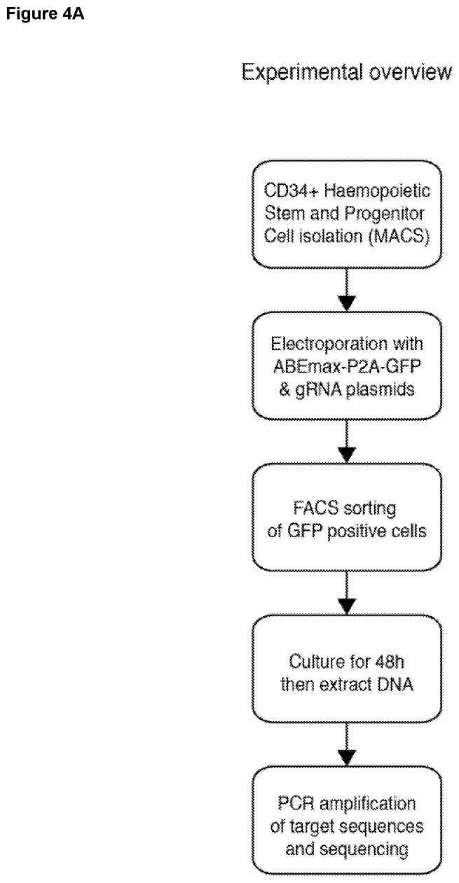

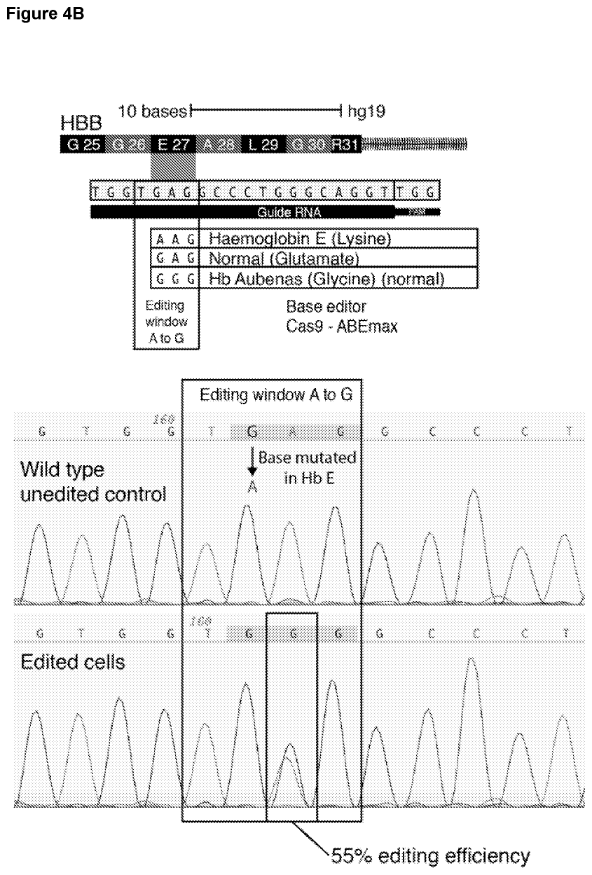

FIG. 4 shows the experimental overview. CD34+ cells were isolated from peripheral blood white cell cones derived from blood donation. These cells were then electroporated with the ABEmax-P2A-GFP plasmid (this was a gift from David Liu Addgene #112101) a separate plasmid to express the guide RNA. GFP positive cells were sorted; cultured for 48h and DNA was extracted and the editing efficiency was assessed by Sanger sequencing.

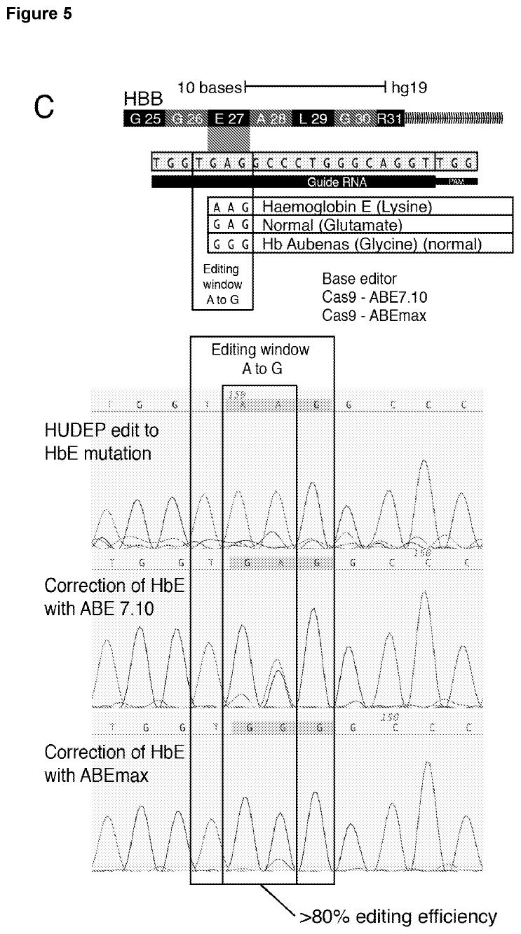

FIG. 5 shows that this strategy is capable of creating over 50% editing of wild type codon 27 from glutamate to glycine (Hb Aubenas). Thus it is highly likely that the adjacent adenine will also be base converted to guanine in haemoglobin E, i.e. that AAG will be converted to GAG or GGG.

### Example 2: Editing of the HbE Variant in HUDEP Cells

Human Umbilical cord blood Derived Erythroid Progenitor (HUDEP) cells serve as a good model for human red blood cell production. The HbE mutation was generated in HUDEP cells using spCas9 ribonuclear protein (RNP) and homologous recombination with a single stranded donor template. Cells were sorted into single cell colonies, expanded and genotyped to give a pure population of homozygous cells with the HbE mutation.

These cells with the HbE mutation were then edited with the ABE 7.10 and the ABEmax base editors using plasmids for the base editors and guide RNAs. A gene editing efficiency of over 80% to Hb Aubenas/WT can be achieved with the ABEmax base editor.

### Example 3: Editing of Human CD34+ Haemopoietic Stem and Progenitor Cells from Patients with HbE Related Thalassaemia

CD34+ cells are isolated from patients with the haemoglobin E mutation using MACS beads (Miltenyi). These cells are edited using the ABE 7.10 and ABEmax base editors using electroporation with a plasmid for the base editor and a plasmid to express the guide RNA.

### Example 4: Editing of Human CD34+ Haemopoietic Stem and Progenitor Cells from Patients with Sickle Cell Disease

CD34+ cells are isolated from patients with the homozygous haemoglobin S mutation using MACS beads (Miltenyi). These cells are edited using the ABE 7.10 and ABEmax base editors using electroporation with a plasmid for the base editor and a plasmid to express the guide RNA.

### Example 5: Editing of Haemopoietic Stem Cells from Patients with HbE-Beta0 Thalassaemia

Patient-derived CD34+ cells were incubated with two plasmids, the base editor containing plasmid ABEmax and a plasmid containing the gRNA and green fluorescent protein (GFP). The cells were electroporated and cultured for 24 h. They were subsequently sorted for GFP positive cells. These cells were cultured for another few days prior to harvesting for DNA. The HBB gene was amplified using PCR and the sequence obtained using high throughput sequencing.

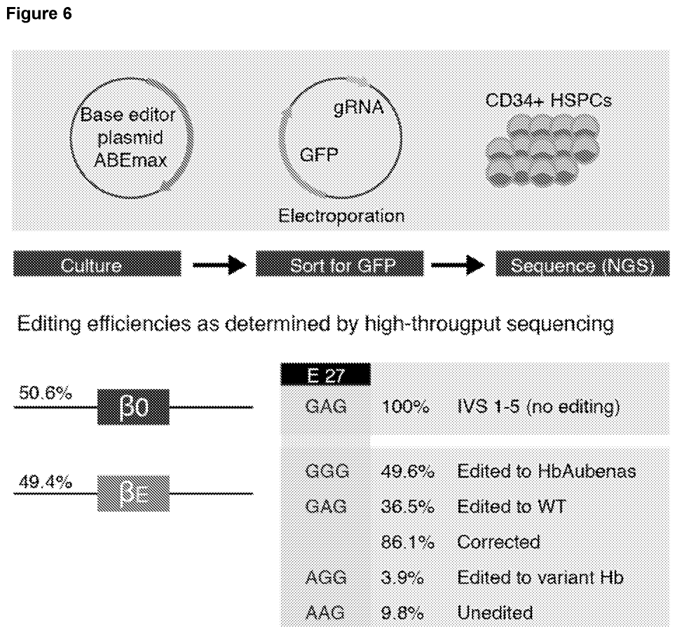

The results are shown in FIG. 6. This shows that the thalassaemic allele was unedited, but that the beta E allele was converted to the sequence for Hb Aubenas in 49.6% of alleles and WT in 36.5%. 3.9% were converted to a previously-undescribed Hb variant.

### REFERENCES

BLACKWELL, R. Q., OEMIJATI, S., PRIBADI, W., WENG, M. I. & LUI, C. S. 1970. Hemoglobin-G Makassar—Beta6 Glu-!Ala. Biochimica Et Biophysica Acta, 214, 396-+.
GAUDELLI, N. M., KOMOR, A. C., REES, H. A., PACKER, M. S., BADRAN, A. H., BRYSON, D. I. & LIU, D. R. 2017. Programmable base editing of A.T to G.C in genomic DNA without DNA cleavage. Nature, 551, 464-+.
HU, J. H., MILLER, S. M., GEURTS, M. H., TANG, W. X., CHEN, L. W., SUN, N., ZEINA, C. M., GAO, X., REES, H. A., LIN, Z. & LIU, D. R. 2018. Evolved Cas9 variants with broad PAM compatibility and high DNA specificity. Nature, 556, 57-+.
JINEK, M., CHYLINSKI, K., FONFARA, I., HAUER, M., DOUDNA, J. A. & CHARPENTIER, E. 2012. A Programmable Dual-RNA-Guided DNA Endonuclease in Adaptive Bacterial Immunity. Science, 337, 816-821.
KOBLAN, L. W., DOMAN, J. L., WILSON, C., LEVY, J. M., TAY, T., NEWBY, G. A., MAIANTI, J. P., RAGURAM, A. & LIU, D. R. 2018. Improving cytidine and adenine base editors by expression optimization and ancestral reconstruction. Nat Biotechnol.
KOMOR, A. C., KIM, Y. B., PACKER, M. S., ZURIS, J. A. & LIU, D. R. 2016. Programmable editing of a target base in genomic DNA without double-stranded DNA cleavage. Nature, 533, 420-4.
KOMOR, A. C., ZHAO, K. T., PACKER, M. S., GAUDELLI, N. M., WATERBURY, A. L., KOBLAN, L. W., KIM, Y. B., BADRAN, A. H. & LIU, D. R. 2017. Improved base excision repair inhibition and bacteriophage Mu Gam protein yields C:G-to-T:A base editors with higher efficiency and product purity. Sci Adv, 3, eaao4774.
KURITA, R., SUDA, N., SUDO, K., MIHARADA, K., HIROYAMA, T., MIYOSHI, H., TANI, K. & NAKAMURA, Y. 2013. Establishment of immortalized human erythroid progenitor cell lines able to produce enucleated red blood cells. PLoS One, 8, e59890.
LACAN, P., FRANCINA, A., PROME, D., DELAUNAY, J., GALACTEROS, F. & WAJCMAN, H. 1996. Hb Aubenas [beta 26(B8)Glu->Gly]: A new variant normally synthesized, affecting the same codon as in Hb E. Hemoglobin, 20, 113-124.
METTANANDA, S., FISHER, C. A., HAY, D., BADAT, M., QUEK, L., CLARK, K., HUBLITZ, P., DOWNES, D., KERRY, J., GOSDEN, M., TELENIUS, J., SLOANE-STANLEY, J. A., FAUSTINO, P., COELHO, A., DOONDEEA, J., USUKHBAYAR, B., SOPP, P., SHARPE, J. A., HUGHES, J. R., VYAS, P., GIBBONS, R. J. & HIGGS, D. R. 2017. Editing an alpha-globin enhancer in primary human hematopoietic stem cells as a treatment for beta-thalassemia. Nat Commun, 8, 424.
NISHIDA, K., ARAZOE, T., YACHIE, N., BANNO, S., KAKIMOTO, M., TABATA, M., MOCHIZUKI, M., MIYABE, A., ARAKI, M., HARA, K. Y., SHIMATANI, Z. & KONDO, A. 2016. Targeted nucleotide editing using hybrid prokaryotic and vertebrate adaptive immune systems. Science, 353.
OLD, J., HARTEVELD, C. L., TRAEGER-SYNODINOS, J., PETROU, M., ANGASTINIOTIS, M. & GALANELLO, R. 2012. In: ND (ed.) Prevention of Thalassaemias and Other Haemoglobin Disorders: Volume 2: Laboratory Protocols. Nicosia, Cyprus.
TRAKARNSANGA, K., GRIFFITHS, R. E., WILSON, M. C., BLAIR, A., SATCHWELL, T. J., MEINDERS, M., COGAN, N., KUPZIG, S., KURITA, R., NAKAMURA, Y., TOYE, A. M., ANSTEE, D. J. & FRAYNE, J. 2017. An immortalized adult human erythroid line facilitates sustainable and scalable generation of functional red cells. Nat Commun, 8, 14750.
VIPRAKASIT, V., WIRIYASATEINKUL, A., SATTAYASEVANA, B., MILES, K. L. & LAOSOMBAT, V. 2002. Hb G-Makassar [beta 6(A3)Glu->Ala; codon 6 (GAG->GCG)]: Molecular characterization, clinical, and hematological effects. Hemoglobin, 26, 245-253.

SEQUENCES

SEQ ID NO: 1
Genomic DNA sequence of the wild-type human HBB
gene (excluding 5′UTR)
ATGGTGCATCTGACTCCTGAGGAGAAGTCTGCCGTTACTGCCCTGTGGGG
CAAGGTGAACGTGGATGAAGTTGGTGGTGAGGCCCTGGGCAGGTTGGTAT
CAAGGTTACAAGACAGGTTTAAGGAGACCAATAGAAACTGGGCATGTGGA
GACAGAGAAGACTCTTGGGTTTCTGATAGGCACTGACTCTCTCTGCCTAT
TGGTCTATTTTCCCACCCTTAGGCTGCTGGTGGTCTACCCTTGGACCCAG
AGGTTCTTTGAGTCCTTTGGGGATCTGTCCACTCCTGATGCTGTTATGGG
CAACCCTAAGGTGAAGGCTCATGGCAAGAAAGTGCTCGGTGCCTTTAGTG
ATGGCCTGGCTCACCTGGACAACCTCAAGGGCACCTTTGCCACACTGAGT
GAGCTGCACTGTGACAAGCTGCACGTGGATCCTGAGAACTTCAGGGTGAG
TCTATGGGACGCTTGATGTTTTCTTTCCCCTTCTTTTCTATGGTTAAGTT
CATGTCATAGGAAGGGGATAAGTAACAGGGTACAGTTTAGAATGGGAAAC
AGACGAATGATTGCATCAGTGTGGAAGTCTCAGGATCGTTTTAGTTTCTT
TTATTTGCTGTTCATAACAATTGTTTTCTTTTGTTTAATTCTTGCTTTCT
TTTTTTTTCTTCTCCGCAATTTTTACTATTATACTTAATGCCTTAACATT
GTGTATAACAAAAGGAAATATCTCTGAGATACATTAAGTAACTTAAAAAA
AAACTTTACACAGTCTGCCTAGTACATTACTATTTGGAATATATGTGTGC
TTATTTGCATATTCATAATCTCCCTACTTTATTTTCTTTTATTTTTAATT
GATACATAATCATTATACATATTTATGGGTTAAAGTGTAATGTTTTAATA
TGTGTACACATATTGACCAAATCAGGGTAATTTTGCATTTGTAATTTTAA
AAAATGCTTTCTTCTTTTAATATACTTTTTTGTTTATCTTATTTCTAATA
CTTTCCCTAATCTCTTTCTTTCAGGGCAATAATGATACAATGTATCATGC
CTCTTTGCACCATTCTAAAGAATAACAGTGATAATTTCTGGGTTAAGGCA
ATAGCAATATCTCTGCATATAAATATTTCTGCATATAAATTGTAACTGAT
GTAAGAGGTTTCATATTGCTAATAGCAGCTACAATCCAGCTACCATTCTG
CTTTTATTTTATGGTTGGGATAAGGCTGGATTATTCTGAGTCCAAGCTAG
GCCCTTTTGCTAATCATGTTCATACCTCTTATCTTCCTCCCACAGCTCCT
GGGCAACGTGCTGGTCTGTGTGCTGGCCCATCACTTTGGCAAAGAATTCA
CCCCACCAGTGCAGGCTGCCTATCAGAAAGTGGTGGCTGGTGTGGCTAAT
GCCCTGGCCCACAAGTATCACTAAGCTCGCTTTCTTGCTGTCCAATTTCT
ATTAAAGGTTCCTTTGTTCCCTAAGTCCAACTACTAAACTGGGGGATATT
ATGAAGGGCCTTGAGCATCTGGATTCTGCCTAATAAAAAACATTTATTTT
CATTGC
SEQ ID NO: 2
Amino acid sequence of the wild-type human HBB
polypeptide.
MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH
SEQ ID NOs: 3 and 4
gRNA genomic target: TGGTAAGGCCCTGGGCAGGT
RNA sequence: UGGUAAGGCCCUGGGCAGGU
SEQ ID NOs: 5 and 6
gRNA genomic target: TTCTCCACAGGAGTCAGATG
RNA sequence: UUCUCCACAGGAGUCAGAUG
SEQ ID NOs: 7 and 8
gRNA genomic target: TTCTCCACAGGAGTCAGGTG
RNA sequence: UUCUCCACAGGAGUCAGGUG
SEQ ID NOs: 9 and 10
gRNA genomic target: AGATGCACCATGGTGTCTGT
RNA sequence: AGAUGCACCAUGGUGUCUGU
SEQ ID NOs: 11 and 12
gRNA genomic target: AGGTGCACCATGGTGTCTGT
RNA sequence: AGGUGCACCAUGGUGUCUGU
SEQ ID NOs: 13 and 14
gRNA genomic target: TCCTAAGGAGAAGTCTGCCG
RNA sequence: UCCUAAGGAGAAGUCUGCCG
SEQ ID NO: 15
5′-end of genomic DNA sequence of the wild-type
human HBB gene covering potential gRNAs
for editing HbS:
GACTTCTCCACAGGAGTCAGATGCACCAT
SEQ ID NO: 16
Genomic DNA sequence of the wild-type human HBB
gene covering potential gRNAs for editing HbE:
GAAGGTGGTAAGGCCCTGGGCAGGTTGGT
SEQ ID NO: 17
Genomic DNA sequence of the wild-type human HBB
gene covering potential gRNAs for editing HbC:
CTGACTCCTAAGGAGAAGTCTGCCGTTAC

1. A process for producing a modified nucleic acid molecule, the process comprising the steps:
(a) contacting a nucleic acid molecule comprising a mutant HBB gene encoding a mutant Hb-β polypeptide with a base editor, wherein the mutant HBB gene comprises a first non-wild-type codon coding for a first non-wild-type amino acid; and
(b) incubating the mutant HBB gene and base editor under conditions such that the base editor is targeted to the nucleotide sequence of the first non-wild-type codon and wherein the base editor edits one or more nucleotides in the first non-wild-type codon to produce a second non-wild-type codon which codes for a second non-wild-type amino acid, thereby producing a modified nucleic acid molecule comprising an edited HBB gene which encodes an edited Hb-β polypeptide,
wherein the edited Hb-β polypeptide has a non-wild-type, yet phenotypically-viable, amino acid sequence.

2. A process as claimed in claim 1, wherein:
Step (a) comprises contacting a nucleic acid molecule comprising a mutant HBB gene encoding a mutant Hb-β polypeptide with a base editor and a gRNA, wherein the mutant HBB gene comprises a first non-wild-type codon coding for a first non-wild-type amino acid and wherein the gRNA is capable of targeting the base editor to the nucleotide sequence of the first non-wild-type codon of the mutant HBB gene; and
Step (b) comprises incubating the mutant HBB gene, base editor and gRNA under conditions such that the gRNA targets the base editor to the nucleotide sequence of the first non-wild-type codon and wherein the base editor edits one or more nucleotides in the first non-wild-type codon to produce a second non-wild-type codon which codes for a second non-wild-type amino acid, thereby producing a modified nucleic acid molecule comprising an edited HBB gene.

3. A process as claimed in claim 1, wherein the HBB gene is a mammalian gene, or a human gene.

4. The process as claimed in claim 1, wherein mutant HBB gene consists of or comprises a nucleotide sequence which encodes an amino acid sequence having 90-99.5%, or 95-99.5% sequence identity to SEQ ID NO: 2.

5. The process as claimed in claim 4, wherein the nucleotide sequence at the codon which corresponds to codon 7 in SEQ ID NO: 1 codes for lysine or valine; and/or the nucleotide sequence at the codon which corresponds to codon 27 in SEQ ID NO: 1 codes for lysine.

6. The process as claimed in claim 5, wherein the nucleotide sequence at the first non-wild-type codon which corresponds to codon 7 in SEQ ID NO: 1 is AAG or GTG; and/or the nucleotide sequence at the first non-wild-type codon which corresponds to codon 27 in SEQ ID NO: 1 is AAG.

7. The process as claimed in wherein the base editor is a programmable nucleic acid binding protein (or an impaired CRISPR-Cas9 mutant, which is capable of being targeted to a target DNA sequence.

8. The process as claimed in claim 7, wherein the base editor is an adenine deaminating editor.

9. The process as claimed in claim 2, wherein:
(i) the guide RNA sequence for editing codon 7 is an 18-22 nucleotide guide RNA which is complementary to a nucleotide sequence located in SEQ ID NO: 15, wherein the wild-type complement of codon 7 (CTC) is replaced by CAC; or
(ii) the guide RNA sequence for editing codon 27 is an 18-22 nucleotide guide RNA which is located in SEQ ID NO: 16, wherein the wild-type codon 27 (GAG) is replaced by AAG; or
(iii) the guide RNA sequence for editing codon 7 is an 18-22 nucleotide guide RNA which is located in SEQ ID NO: 17, wherein the wild-type of codon 7 (GAG) is replaced by AAG.

10. The process as claimed in wherein the edited HBB gene consists of or comprises:
(i) a nucleotide sequence having 90-99.9% nucleotide sequence identity to SEQ ID NO: 1 or a nucleotide sequence encoding an amino acid sequence having 95-99.5% amino acid sequence identity to SEQ ID NO: 2; and wherein
(ii) the nucleotide sequence at the codon which corresponds to codon 7 in SEQ ID NO: 1 codes for glycine or alanine; and/or the nucleotide sequence at the codon which corresponds to codon 27 in SEQ ID NO: 1 codes for glycine.

11. The process as claimed in claim 1, wherein:
(i) the position of the first non-wild-type codon is codon 7, the wild-type codon at this position is GAG (glutamate), the first non-wild-type (mutant) codon sequence is AAG (lysine), the base editor is an adenine base editor and the second non-wild-type codon is GGG (glycine); or
(ii) the position of the first non-wild-type codon is codon 7, the wild-type codon at this position is GAG (glutamate), the first non-wild-type (mutant) codon sequence is GTG (valine), the base editor is an adenine base editor and the second non-wild-type codon is GCG (alanine); or
(iii) the position of the first non-wild-type codon is codon 27, the wild-type codon at this position is GAG (glutamate), the first non-wild-type (mutant) codon sequence is AAG (lysine), the base editor is an adenine base editor and the second non-wild-type codon is GGG (glycine).

12. The process as claimed in claim 1, which additionally includes, prior to Step (a), the step of obtaining a sample of haematopoietic stem cells from a subject, or from a human subject, wherein the stem cells comprise nucleic acid molecules comprising mutant HBB genes.

13. The process as claimed in claim 1, which additionally comprises, prior to Step (a), the step of modifying the nucleotide sequences of one or more PAM sites in the vicinity of the first non-wild-type codon in order to increase efficiency of the base-editing process.

14. The process as claimed in claim 1, wherein the process is performed on haematopoietic stem cells which have previously been obtained from a first subject, or from a human subject, wherein the stem cells comprise nucleic acid molecules comprising mutant HBB genes.

15. The process as claimed in claim 14, the process additionally comprises the subsequent step of introducing a population of haematopoietic stem cells comprising modified nucleic acid molecules comprising edited HBB genes, optionally after expansion of the cells, into a second subject, wherein the first and second subjects are the same or related subjects.

16. A population of isolated cells comprising haematopoietic stem cells or progenitor cells comprising edited HBB genes, the edited HBB genes comprising:
(i) a nucleotide sequence having 90-99.9% nucleotide sequence identity to SEQ ID NO: 1 or a nucleotide sequence encoding an amino acid sequence having 95-99.5% amino acid sequence identity to SEQ ID NO: 2; and wherein
(ii) the nucleotide sequence at the codon which corresponds to codon 7 in SEQ ID NO: 1 codes for glycine or alanine; and/or the nucleotide sequence at the codon which corresponds to codon 27 in SEQ ID NO: 1 codes for glycine.

17. The population of isolated cells as claimed in claim 16, wherein the population of isolated cells comprises at least 20% haematopoietic stem cells or progenitor cells having modified nucleic acid molecules comprising edited HBB genes, or at least 40%, at least 60%, at least 80% or 100% haematopoietic stem cells or progenitor cells having modified nucleic acid molecules comprising edited HBB genes.
